(19) United States
(12) Patent Application Publication
(10) Pub. No.: US 2026/0363556 A1
(43) Pub. Date: Nov. 19, 2026

(54) WRIST-WORN VASCULAR STIFFNESS DETECTOR

(71) Applicant: Beihang University, Beijing (CN)

(72) Inventors: Shuqi REN, Beijing (CN); Anqiang SUN, Beijing (CN); Li WANG, Beijing (CN); Yubo FAN, Beijing (CN); Xiaoyan DENG, Beijing (CN)

(21) Appl. No.: 19/226,767

(22) Filed: Jun. 3, 2025

(30) Foreign Application Priority Data
May 16, 2025 (CN) 202510632243.5

(51) Int. Cl. A61B 5/02; A61B 5/00; A61B 5/022; G05B 17/02

(52) U.S. Cl. CPC A61B 5/02007; A61B 5/002; A61B 5/02233; A61B 5/681; A61B 5/7203; A61B 5/742; A61B 5/7455; A61B 5/748; G05B 17/02; A61B 2562/0219; A61B 2562/0247; A61B 2562/06

(57) ABSTRACT

Provided is a wrist-worn vascular stiffness detector, which relates to the technical field of medical devices. The wrist-worn vascular stiffness detector includes a wristband and a detection case connected to the wristband, where an air bag is provided at an inner side of the wristband; a vibration sensor for acquiring a vascular vibration signal is provided in the air bag; and a pressure sensor is provided at a bottom of the detection case. Worn on a wrist of a user, the wrist-worn vascular stiffness detector is configured to control, according to the pressure signal detected by the pressure sensor and/or the vascular vibration signal detected by the vibration sensor, the air bag to inflate and deflate, acquire the vascular vibration signal in the target time period when the air bag is deflated, and calculate vascular stiffness based on the vascular vibration signal in the target time period.

### CROSS REFERENCE TO RELATED APPLICATION

This patent application claims the benefit and priority of Chinese Patent Application No. 202510632243.5, filed with the China National Intellectual Property Administration on May 16, 2025, the disclosure of which is incorporated by reference herein in its entirety as part of the present application.

### TECHNICAL FIELD

The present disclosure relates to the technical field of medical devices, and in particular to a wrist-worn vascular stiffness detector.

### BACKGROUND

With the intensification of population aging, the morbidity and mortality of cardiovascular diseases (CVDs) will rise further, and there is a growing demand for early screening of the CVDs. Arterial stiffness is an independent predictor for the CVDs. Existing non-invasive detection methods for the arterial stiffness are primarily based on a pulse wave velocity (PWV). However, for the need of bulky instruments, reliance on hospitals and physical examination centers, complex operation with strong specialized expertise, and high cost, these methods are restricted to detect the vascular stiffness at home. In view of principles for measuring the vascular stiffness, the PWV-based methods are hardly applied to portable devices.

### SUMMARY

An objective of the present disclosure is to provide a wrist-worn vascular stiffness detector. The wrist-worn vascular stiffness detector is easy-to-operate, portable, and capable of acquiring a health state of the blood vessel the user at any time in different environments.

To achieve the above objective, the present disclosure provides the following solutions.

According to a first aspect, the present disclosure provides a wrist-worn vascular stiffness detector, including a wristband, an air bag, a pressure sensor, a vibration sensor, and a detection case, where the air bag, the vibration sensor and the detection case are provided on the wristband; and the pressure sensor is provided at a bottom of the detection case;
an air pump, an air valve and a mainboard are provided in the detection case; and the air pump is connected to the air bag through the air valve;
the vibration sensor and the pressure sensor are connected to the mainboard; and the mainboard is further connected to a control end of the air pump and a control end of the air valve;
in response to use, the wristband is wound on a wrist of a user, the air bag and the vibration sensor are located between the wristband and the wrist of the user, and the pressure sensor is located between the detection case and the wrist of the user; the vibration sensor is configured to acquire a vascular vibration signal of the user, and transmit the vascular vibration signal to the mainboard; and the pressure sensor is configured to detect a pressure signal, and transmit the pressure signal to the mainboard; and
the mainboard is configured to control, based on the vascular vibration signal and/or the pressure signal, the air valve and the air pump to inflate and deflate the air bag, extract a vascular vibration signal in a target time period, and calculate vascular stiffness according to the vascular vibration signal in the target time period; and the target time period is a deflation time period, namely a time period between complete collapse of a blood vessel and complete restoration of the blood vessel.

Optionally, in terms of controlling, based on the vascular vibration signal, the air valve and the air pump to inflate and deflate the air bag, the mainboard is specifically configured to:
control, when receiving a vascular stiffness detection command, the air valve and the air pump to inflate the air bag;
control, within a preset time period after the vascular vibration signal and/or the pressure signal characterize the complete collapse of the blood vessel during inflation, the air valve and the air pump to deflate the air bag; and
control, when the vascular vibration signal and/or the pressure signal characterize the complete restoration of the blood vessel during deflation, the air valve and the air pump to stop deflating the air bag.

Optionally, in terms of calculating the vascular stiffness according to the vascular vibration signal in the target time period, the mainboard is specifically configured to:
extract one or more target time and frequency domain features of the vascular vibration signal in the target time period, the target time and frequency domain feature being a timing feature significantly correlated with the vascular stiffness; and
calculate the vascular stiffness with a vascular stiffness calculation model according to the target time and frequency domain feature, the vascular stiffness calculation model being configured to characterize a relationship between the target time and frequency domain feature and the vascular stiffness.

Optionally, before extracting the one or more target time and frequency domain features of the vascular vibration signal, the mainboard is further configured to:
perform detrending, denoising and smoothing on the vascular vibration signal.

Optionally, the target time and frequency domain features include one or more of an absolute maximum amplitude, a center of mass, a skewness, a waveform index, a center of mass frequency, a frequency corresponding a maximum power value, an inherent frequency, and an average frequency.

Optionally, the center of mass is calculated by:

$$m = \frac{{a}_{1} \cdot {x}_{1} + {a}_{2} \cdot {x}_{2} + … + {a}_{n} \cdot {x}_{n}}{{a}_{1} + {a}_{2} + … + {a}_{n}} ,$$

where, m is the center of mass, x₁, x₂ and xₙ are respectively a coordinate of a first frame, a coordinate of a second frame and a coordinate of an nth frame of the vascular vibration signal in a target coordinate system, the target coordinate system being a coordinate system established according to a position of each frame of the vascular vibration signal, a₁, a₂ and aₙ are respectively a maximum amplitude of the first frame, a maximum amplitude of the second frame and a maximum amplitude of the nth frame of the vascular vibration signal, and n is a number of frames of the vascular vibration signal in the target time period.

Optionally, the wrist-worn vascular stiffness detector further includes a step-counting three-dimensional (3D) sensor;
the step-counting 3D sensor is provided in the detection case; the step-counting 3D sensor is connected to the mainboard; and the mainboard is further configured to count a number of steps according to a detection signal of the step-counting 3D sensor; and
the mainboard is further configured to acquire, when the vascular vibration signal or the pressure signal characterizes the collapse of the blood vessel, a pressure signal detected by the pressure sensor, to serve as a first wristband pressure; and acquire, when the vascular vibration signal or the pressure signal characterizes the complete restoration of the blood vessel, a pressure signal detected by the pressure sensor, to serve as a second wristband pressure.

Optionally, the wrist-worn vascular stiffness detector further includes an accelerometer; the accelerometer is provided in the detection case; the accelerometer is connected to the mainboard; and the mainboard is further configured to recognize a movement state of the user based on an acceleration signal detected by the accelerometer, ensuring that the user is in a resting state during measurement.

Optionally, the wrist-worn vascular stiffness detector further includes a motor vibrator; the motor vibrator is provided in the detection case; a control end of the motor vibrator is connected to the mainboard; and the mainboard is further configured to control, when the vascular stiffness meets an early warning condition, the motor vibrator to vibrate.

Optionally, the mainboard includes an operation module, a Bluetooth module, a power management module, a touch control chip, a storage module, and a display module;
the Bluetooth module, the touch control chip, the storage module and the display module are connected to the operation module; and
the power management module is connected to the operation module, the Bluetooth module, the touch control chip, the storage module and the display module.

According to specific embodiments of the present disclosure, the present disclosure has the following technical effects.

According to the wrist-worn vascular stiffness detector provided by the present disclosure, the wrist-worn vascular stiffness detector includes the wristband and the detection case connected to the wristband. The air bag is provided at an inner side of the wristband. The vibration sensor for acquiring the vascular vibration signal is provided in the air bag. The pressure sensor is provided at the bottom of the detection case. Worn on a wrist of a user, the wrist-worn vascular stiffness detector is configured to control, according to the pressure signal detected by the pressure sensor and the vascular vibration signal detected by the vibration sensor, the air bag to inflate and deflate, acquire the vascular vibration signal in the target time period when the air bag is deflated, and calculate vascular stiffness based on the vascular vibration signal in the target time period. Only with the vibration sensor and the mainboard, the wrist-worn vascular stiffness detector provided by the present disclosure is easy-to-operate, portable, and capable of acquiring a health state of the blood vessel of the user at any time in different environments.

### BRIEF DESCRIPTION OF THE DRAWINGS

To describe the technical solutions in the embodiments of the present disclosure or in the prior art more clearly, the following briefly describes the accompanying drawings required for the embodiments. Apparently, the accompanying drawings in the following description show merely some embodiments of the present disclosure, and a person of ordinary skill in the art may still derive other accompanying drawings from these accompanying drawings without creative efforts.

FIG. 1 is a structure diagram of a wrist-worn vascular stiffness detector according to an embodiment of the present disclosure;

FIG. 2 is a structure diagram of a detection case according to an embodiment of the present disclosure;

FIG. 3 is a structure diagram of a mainboard according to an embodiment of the present disclosure;

FIG. 4 is a schematic structural diagram of a wrist-worn vascular stiffness detector according to an embodiment of the present disclosure;

FIGS. 5A-5B illustrate comparison between an original vascular vibration signal and a preprocessed vascular vibration signal according to an embodiment of the present disclosure; and

FIG. 6 illustrates a statistical result of inherent frequencies of vascular vibration signals from an arterial stiffness group and a control group according to an embodiment of the present disclosure.

### REFERENCE NUMERALS

1: detection case, 11: pressure sensor, 12: step-counting 3D sensor, 13: mainboard, 14: battery, 15: air pump, 16: air valve, 2: wristband, 21: air bag, 22: vibration sensor, 131: Bluetooth module, 132: digital signal processor, 133: random access memory (RAM), 134: flash memory, 135: power management module, and 136: motor vibrator.

### DETAILED DESCRIPTION OF THE EMBODIMENTS

The technical solutions in the embodiments of the present disclosure are clearly and completely described below with reference to the drawings in the embodiments of the present disclosure. Apparently, the described embodiments are only some rather than all of the embodiments of the present disclosure. All other embodiments obtained by a person of ordinary skill in the art based on the embodiments of the present disclosure without creative efforts shall fall within the protection scope of the present disclosure.

To make the above objectives, features, and advantages of the present disclosure more obvious and easy to understand, the present disclosure will be further described below in detail with reference to the accompanying drawings and specific implementations.

Korotkoff sounds are the sounds heard through a stethoscope when blood vessels are squeezed by the cuff to collapse during blood pressure measurement, and have been widely applied to non-invasive blood pressure measurement. The Korotkoff sounds come from vibration of blood vessels and certainly include information about stiffening of the blood vessels. Besides, ease of operation, portability and intellectualization have become main themes of the modern society, and the demand for early screening of the CVDs is huge.

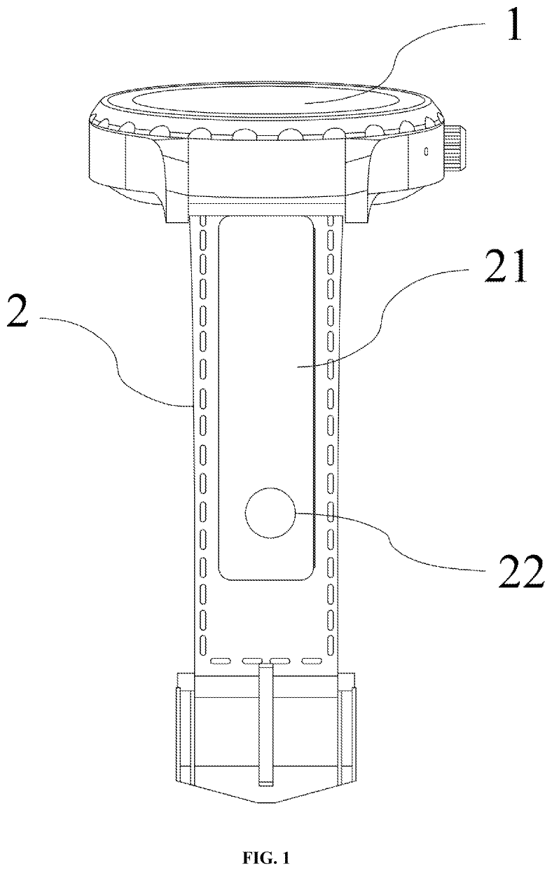

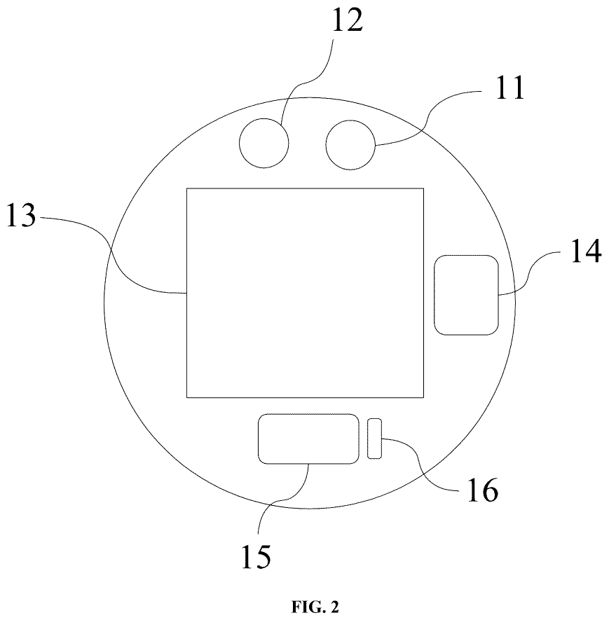

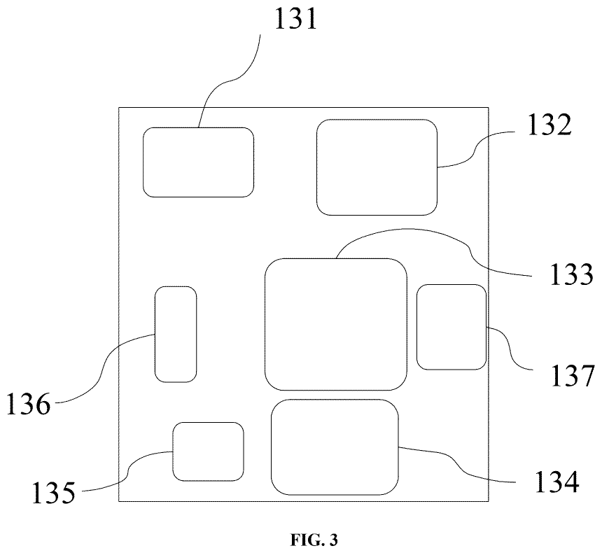

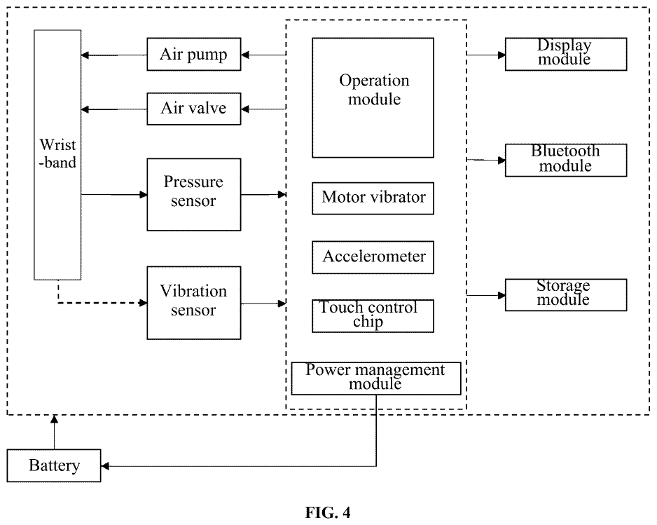

In an exemplary embodiment, a wrist-worn vascular stiffness detector is provided. As shown in FIG. 1 to FIG. 4, the wrist-worn vascular stiffness detector includes a wristband 2, an air bag 21, a vibration sensor 22, a pressure sensor 11, and a detection case 1. The air bag 21, the vibration sensor 22 and the detection case 1 are provided on the wristband 2. The pressure sensor 11 is provided at a bottom of the detection case 1. An air pump 15, an air valve 16 and a mainboard 13 are provided in the detection case 1. The air pump 15 is connected to the air bag 21 through the air valve 16. The vibration sensor 22 and the pressure sensor 11 are connected to the mainboard 13. The mainboard 13 is further connected to a control end of the air pump 15 and a control end of the air valve 16. In response to use, the wristband 2 is wound on a wrist of a user, the air bag 21 and the vibration sensor 22 are located between the wristband 2 and the wrist of the user, and the pressure sensor 11 is located between the detection case 1 and the wrist of the user. The vibration sensor 22 is configured to acquire a vascular vibration signal of the user, and transmit the vascular vibration signal to the mainboard. The pressure sensor 11 is configured to detect a pressure signal, and transmit the pressure signal to the mainboard 13. The mainboard 13 is configured to control, based on the vascular vibration signal and/or the pressure signal, the air valve 16 and the air pump 15 to inflate and deflate the air bag 21, extract a vascular vibration signal in a target time period, and calculate vascular stiffness according to this signal. The target time period is a deflation time period, namely a time period between complete collapse of a blood vessel and complete restoration of the blood vessel.

According to the vascular stiffness detector provided by the present disclosure, the wristband 2 can be worn on the wrist. The blood vessel is squeezed through inflation and deflation of the air bag 21. The vascular vibration signal of the radial artery is captured through the vibration sensor 22. The degree of stiffening on the blood vessel of the human body is detected through processing and feature extraction on the vascular vibration signal. The mainboard 13 is configured to send a command to the air pump 15 and the air valve 16, thereby controlling the air pump 15 to inflate and deflate the air bag 21. This specifically includes the following steps:

When receiving a vascular stiffness detection command, the mainboard controls the air valve and the air pump to inflate the air bag. Within a preset time period after the vascular vibration signal and/or the pressure signal characterize the complete collapse of the blood vessel during inflation, the mainboard controls the air valve and the air pump to deflate the air bag. When the vascular vibration signal and/or the pressure signal characterize the complete restoration of the blood vessel during deflation, the mainboard controls the air valve and the air pump to stop deflating the air bag.

The collapse refers to a process, during which vibration signals appear from initial collapse to complete collapse, but disappear after the complete collapse. Conversely, signals appear during the whole restoration process, but disappear after the complete restoration. Hence, the complete collapse and the complete restoration can be determined according to the presence of vibration signal. Besides, the complete collapse and the complete restoration of the blood vessel may also be reflected by the pressure signal, so the complete collapse and the complete restoration may also be determined according to the pressure signal.

In the embodiment of the present disclosure, whether the blood vessel is collapsed completely is determined by detecting whether the vascular vibration signal disappears. Alternatively, whether the blood vessel is collapsed completely is determined by detecting whether an amplitude of the pressure signal is greater than a threshold. Whether the blood vessel is restored completely is determined by detecting whether the vascular vibration signal disappears again. Alternatively, whether the blood vessel is restored completely is determined by detecting whether the amplitude of the pressure signal is less than the threshold. Generally, when the air bag is deflated completely or an internal pressure and an external pressure of the air bag are the same, it is considered that the blood vessel is restored completely.

In another exemplary embodiment, the mainboard 13 is further configured to acquire, through the pressure sensor 11, wristband pressures when the blood vessel precisely reaches the complete collapse and the complete restoration, namely a first wristband pressure and a second wristband pressure, for storage and operation.

In another exemplary embodiment, the wrist-worn vascular stiffness detector further includes a motor vibrator 136. A control end of the motor vibrator 136 is connected to the mainboard 13. The motor vibrator 136 is configured to drive, with a magnetic field generated by a current through electromagnetic induction, a rotor to rotate, thereby generating vibration. It is used to alert abnormal vibration of the device.

In another exemplary embodiment, the wrist-worn vascular stiffness detector further includes an accelerometer. The accelerometer is connected to the mainboard 13. The accelerometer is configured to recognize a movement state of the user, ensuring that the user is in a resting state during measurement.

In another exemplary embodiment, a battery 14 for supplying power to the device is further provided in the detection case. An operation module, a Bluetooth module 131, a power management module 135, a touch control chip 137, a storage module, and a display module are provided on the mainboard 13.

The Bluetooth module 131 is configured to transmit data, such that the first wristband pressure, the second wristband pressure and the vascular vibration signal are displayed at a personal computer (PC) terminal, and an application (APP) terminal. The operation module is configured to process, calculate and control signals acquired by the pressure sensor and the vibration sensor. The storage module includes an RAM and a flash memory, and is configured to store the data. The power management module 135 is configured to manage power supply and power control of the chip, so as to realize low-power operation, thereby prolonging the service life of the battery, and improving the stability and reliability of the system. The touch control chip 137 is configured to realize a touchscreen operation of the device, and transmit the command to the operation module through a touch. The operation module operates according to the above command. The display module is configured to display each functional result.

In another exemplary embodiment, the operation module is realized based on a digital signal processor 132. The vascular vibration signal obtained by the vibration sensor 22 is acquired by a 24-bit analog-to-digital (AD) converter module in the digital signal processor 132, at an acquisition frequency of 1000 Hz. Data acquired by the AD converter module is transmitted to the storage module, such that the digital signal processor 132 invokes the data and completes processing and calculation on the data.

The processing of the digital signal processor 132 includes, but is not limited to, detrending, denoising, smoothing and the like on a Korotkoff signal, so as to improve a signal-to-noise ratio (SNR) of the signal and accuracy of calculation.

The calculation of the digital signal processor 132 is to calculate the vascular stiffness through target time and frequency domain features of the vascular vibration signal. The target time and frequency domain features are significantly correlated with the vascular stiffness, and include, but is not limited to, an absolute maximum amplitude, a center of mass, a skewness, a waveform index, a center of mass frequency, a frequency corresponding a maximum power value, an inherent frequency, an average frequency and the like of the vascular vibration signal. At last, the time and frequency domain features such as the absolute maximum amplitude, the center of mass, the skewness, the waveform index, the center of mass frequency, the frequency corresponding the maximum power value, the inherent frequency, the average frequency and the like of the vascular vibration signal in the target time period are converted into the vascular stiffness.

In another exemplary embodiment, the vascular stiffness is calculated by constructing a vascular stiffness calculation model. The vascular stiffness calculation model may be any one of a neural network model, a linear regression model and the like.

In another exemplary embodiment, the vascular stiffness is calculated with the following steps:

Each target time and frequency domain feature is normalized and nondimensionalized.

According to a correlation between each target time and frequency domain feature and the vascular stiffness, a weight is assigned to the target time and frequency domain feature.

According to the weight of the target time and frequency domain feature, all target time and frequency domain features are integrated into a new stiffening index, namely the vascular stiffness.

The vascular stiffness obtained with the above calculation method is displayed by the display module, stored to the storage module, and output subsequently by the PC terminal or the APP terminal.

In another exemplary embodiment, a center of mass calculation method is proposed. In the embodiment of the present disclosure, a maximum amplitude and a position of the maximum amplitude are taken into account at the same time, which can reflect features of the vascular vibration signal more comprehensively. The vascular vibration signal is framed. With a midpoint of a total frame count n, namely a position of a └n/2┘th or ┌n/2┐th frame of the vascular vibration signal, as an origin, new coordinate axes are established to obtain a target coordinate system. A coordinate for each frame of the vascular vibration signal in the target coordinate system is as follows: x₁, x₂, . . . , xₙ. Meanwhile, a maximum amplitude for each frame of the vascular vibration signal is obtained: a₁, a₂, . . . , aₙ. Coordinate and maximum amplitudes for all frames of the vascular vibration signal are weighted and summed, and the result is divided by the sum of the maximum amplitudes, thereby obtaining the center of mass:

$$m = \frac{{a}_{1} \cdot {x}_{1} + {a}_{2} \cdot {x}_{2} + … + {a}_{n} \cdot {x}_{n}}{{a}_{1} + {a}_{2} + … + {a}_{n}}$$

where, m is the center of mass, x₁, x₂ and xₙ are respectively a coordinate of a first frame, a coordinate of a second frame and a coordinate of an nth frame of the vascular vibration signal in the target coordinate system, the target coordinate system being a coordinate system established according to a position of each frame of the vascular vibration signal, a₁, a₂ and aₙ are respectively a maximum amplitude of the first frame, a maximum amplitude of the second frame and a maximum amplitude of the nth frame of the vascular vibration signal, and n is a number of frames of the vascular vibration signal in the target time period.

In another exemplary embodiment, the digital signal processor is further configured to calculate a blood pressure and a heart rate based on the vascular vibration signal. The blood pressure and the heart rate are also displayed by the display module, stored to the storage module, and output subsequently by the PC terminal or the APP terminal.

In another exemplary embodiment, the Bluetooth module 131 is provided on the mainboard 13 to transmit a calculated result of the digital signal processor 132, data stored in the RAM 133 and data stored in the flash memory 134 to the PC terminal or the APP terminal. Under the control of the digital signal processor 132, the vibration sensor 22 and the pressure sensor 11 acquire a signal in real time. The signal is stored to the flash memory 134, and then invoked by the digital signal processor 132 for processing and calculation. The power management module 135 is configured to effectively allocate electric energy of each component to ensure normal operation and performance optimization of the device. The motor vibrator 136 is configured to drive, with a magnetic field generated by a current through electromagnetic induction, a rotor to rotate, thereby generating vibration. It is used to alert testing personnel with vibration in case of an abnormal measured result. Besides, the touch control chip 137 can provide touchscreen operations for all functions. A touch command is transmitted to the digital signal processor 132, and a functional command is given by the digital signal processor 132 for further operation. The touch control chip 137 facilitates use of the vascular stiffness detector in various scenarios to improve the use experience.

In another exemplary embodiment, the wrist-worn vascular stiffness detector may further be functionally extended. A step-counting 3D sensor 12 is further provided in the detection case 1. When the vascular stiffness detector is worn for walking or movement, the vascular stiffness detector can count the steps, distance and burned calories, providing support for fitness exercises. Certainly, the wrist-worn vascular stiffness detector may also have other extended functions, including blood oxygen monitoring, blood glucose monitoring, electrocardiogram monitoring, sleep monitoring, positioning and navigation, time display, etc.

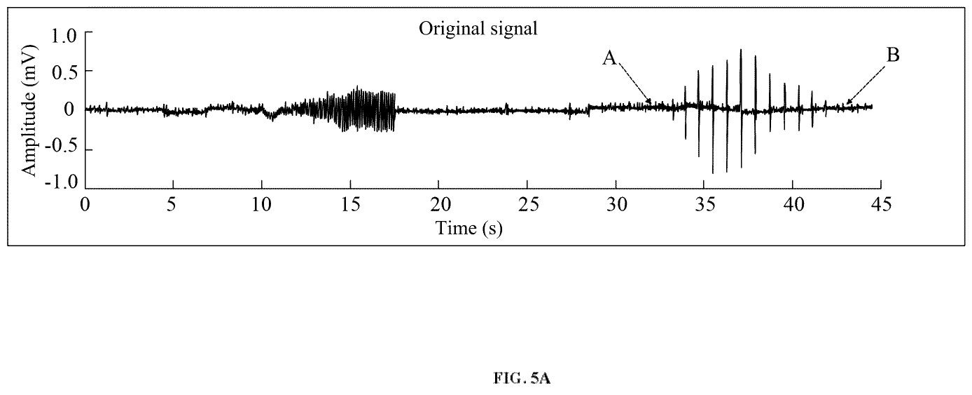

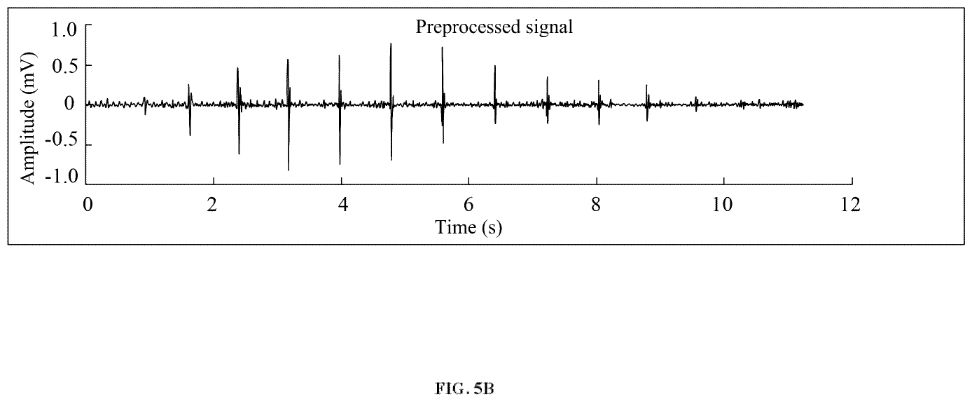

In another exemplary embodiment, referring to FIGS. 5A-5B, vascular vibration signals are respectively acquired from an arterial stiffness group and a control group in the embodiment of the present disclosure. Baseline drift removal and filtration are performed on the signal to improve the SNR of the signal and the accuracy of the result. A detrend function is used to eliminate the direct current (DC) component and the linear trend term. A wavelet transform is used for denoising. According to a law that the intensity of the useful signal is allocated uniformly on each level, while the intensity of the noise is decreased with an increase of the decomposition level, different thresholds are assigned to wavelet coefficients on decomposition levels. At last, the proposed new threshold function is used to denoise the wavelet threshold.

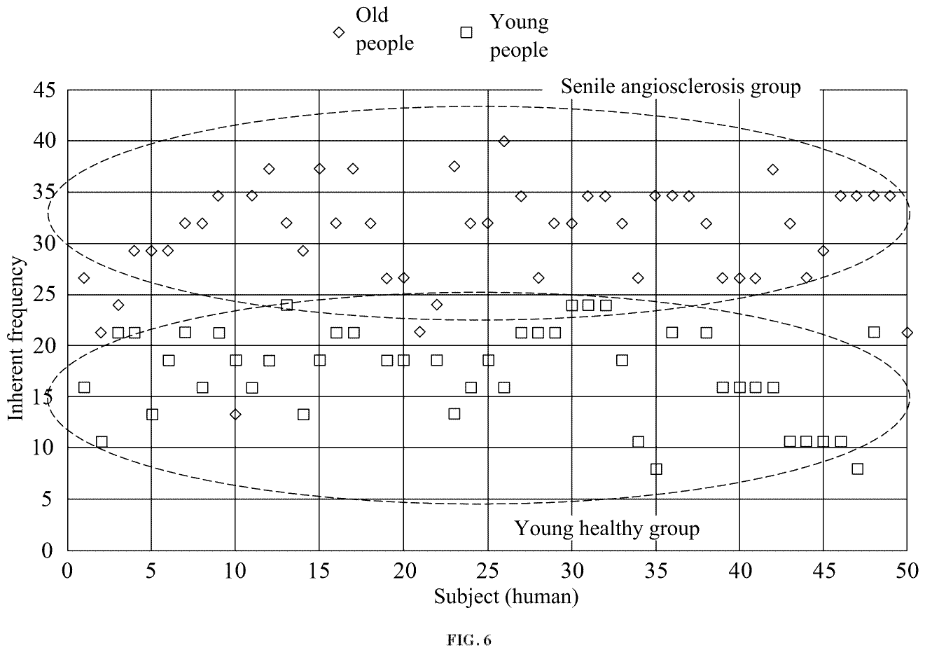

In another exemplary embodiment, referring to FIG. 6, time and frequency domain features, including but not limited to the maximum amplitude, the center of mass, the inherent frequency and the average frequency, are extracted from the preprocessed signal. FIG. 6 illustrates a statistical result of inherent frequencies. According to the result, the arterial stiffness can be recognized through typical time and frequency domain features.

Other signal processing methods or other time and frequency domain features of the vascular vibration signal may also be selected. The embodiment of the present disclosure merely provides a feasible solution, and cannot be constituted as a limitation to the protection scope of the present disclosure.

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of the present disclosure. The description of the foregoing embodiments is used to help illustrate the method of the present disclosure and the core principles thereof. In addition, those of ordinary skill in the art can make various modifications in terms of specific implementations and scope of application in accordance with the teachings of the present disclosure. In conclusion, the content of the present specification shall not be construed as a limitation to the present disclosure.

What is claimed is:

1. A wrist-worn vascular stiffness detector, comprising a wristband, an air bag, a pressure sensor, a vibration sensor, and a detection case, wherein the air bag, the vibration sensor and the detection case are provided on the wristband; and the pressure sensor is provided at a bottom of the detection case;
an air pump, an air valve and a mainboard are provided in the detection case; and the air pump is connected to the air bag through the air valve;
the vibration sensor and the pressure sensor are connected to the mainboard; and the mainboard is further connected to a control end of the air pump and a control end of the air valve;
in response to use, the wristband is wound on a wrist of a user, the air bag and the vibration sensor are located between the wristband and the wrist of the user, and the pressure sensor is located between the detection case and the wrist of the user; the vibration sensor is configured to acquire a vascular vibration signal of the user, and transmit the vascular vibration signal to the mainboard; and the pressure sensor is configured to detect a pressure signal, and transmit the pressure signal to the mainboard; and
the mainboard is configured to control, based on the vascular vibration signal and/or the pressure signal, the air valve and the air pump to inflate and deflate the air bag, extract a vascular vibration signal in a target time period, and calculate vascular stiffness according to the vascular vibration signal in the target time period; and the target time period is a deflation time period, namely a time period between complete collapse of a blood vessel and complete restoration of the blood vessel.

2. The wrist-worn vascular stiffness detector according to claim 1, wherein in terms of controlling, based on the vascular vibration signal, the air valve and the air pump to inflate and deflate the air bag, the mainboard is specifically configured to:
control, when receiving a vascular stiffness detection command, the air valve and the air pump to inflate the air bag;
control, within a preset time period after the vascular vibration signal and/or the pressure signal characterize the complete collapse of the blood vessel during inflation, the air valve and the air pump to deflate the air bag; and
control, when the vascular vibration signal and/or the pressure signal characterize the complete restoration of the blood vessel during deflation, the air valve and the air pump to stop deflating the air bag.

3. The wrist-worn vascular stiffness detector according to claim 1, wherein in terms of calculating the vascular stiffness according to the vascular vibration signal in the target time period, the mainboard is specifically configured to:
extract one or more target time and frequency domain features of the vascular vibration signal in the target time period, the target time and frequency domain feature being a timing feature significantly correlated with the vascular stiffness; and
calculate the vascular stiffness with a vascular stiffness calculation model according to the target time and frequency domain feature, the vascular stiffness calculation model being configured to characterize a relationship between the target time and frequency domain feature and the vascular stiffness.

4. The wrist-worn vascular stiffness detector according to claim 3, wherein before extracting the one or more target time and frequency domain features of the vascular vibration signal, the mainboard is further configured to:
perform detrending, denoising and smoothing on the vascular vibration signal.

5. The wrist-worn vascular stiffness detector according to claim 3, wherein the target time and frequency domain features comprise one or more of an absolute maximum amplitude, a center of mass, a skewness, a waveform index, a center of mass frequency, a frequency corresponding a maximum power value, an inherent frequency, and an average frequency.

6. The wrist-worn vascular stiffness detector according to claim 5, wherein the center of mass is calculated by:
$$m = \frac{{a}_{1} \cdot {x}_{1} + {a}_{2} \cdot {x}_{2} + … + {a}_{n} \cdot {x}_{n}}{{a}_{1} + {a}_{2} + … + {a}_{n}} ,$$
wherein, m is the center of mass, x₁, x₂ and xₙ are respectively a coordinate of a first frame, a coordinate of a second frame and a coordinate of an nth frame of the vascular vibration signal in a target coordinate system, the target coordinate system being a coordinate system established according to a position of each frame of the vascular vibration signal, a₁, a₂ and aₙ are respectively a maximum amplitude of the first frame, a maximum amplitude of the second frame and a maximum amplitude of the nth frame of the vascular vibration signal, and n is a number of frames of the vascular vibration signal in the target time period.

7. The wrist-worn vascular stiffness detector according to claim 1, further comprising a step-counting three-dimensional (3D) sensor, wherein
the step-counting 3D sensor is provided in the detection case; the step-counting 3D sensor is connected to the mainboard; and the mainboard is further configured to count a number of steps according to a detection signal of the step-counting 3D sensor; and
the mainboard is further configured to acquire, when the vascular vibration signal or the pressure signal characterizes the collapse of the blood vessel, a pressure signal detected by the pressure sensor, to serve as a first wristband pressure; and acquire, when the vascular vibration signal or the pressure signal characterizes the complete restoration of the blood vessel, a pressure signal detected by the pressure sensor, to serve as a second wristband pressure.

8. The wrist-worn vascular stiffness detector according to claim 1, further comprising an accelerometer, wherein the accelerometer is provided in the detection case; the accelerometer is connected to the mainboard; and the mainboard is further configured to recognize a movement state of the user based on an acceleration signal detected by the accelerometer, ensuring that the user is in a resting state during measurement.

9. The wrist-worn vascular stiffness detector according to claim 1, further comprising a motor vibrator, wherein the motor vibrator is provided in the detection case; a control end of the motor vibrator is connected to the mainboard; and the mainboard is further configured to control, when the vascular stiffness meets an early warning condition, the motor vibrator to vibrate.

10. The wrist-worn vascular stiffness detector according to claim 1, wherein the mainboard comprises an operation module, a Bluetooth module, a power management module, a touch control chip, a storage module, and a display module;
the Bluetooth module, the touch control chip, the storage module and the display module are connected to the operation module; and
the power management module is connected to the operation module, the Bluetooth module, the touch control chip, the storage module and the display module.
